(19) United States
(12) Patent Application Publication
(10) Pub. No.: US 2022/0054138 A1
(43) Pub. Date: Feb. 24, 2022

(54) TRANS-ESOPHAGEAL AORTIC FLOW RATE CONTROL

(71) Applicant: University of Maryland, Baltimore, Baltimore, MD (US)

(72) Inventors: Joseph Rabin, Silver Spring, MD (US); Zhongjun Wu, Marriottsville, MD (US); Philip Haarstad, Chanhassen, MN (US); David Heisserer, Minneapolis, MN (US); Steve Roller, Minneapolis, MN (US); Jesse Haworth, North Barrington, IL (US); Kelly Jensen, North Barrington, IL (US); Andy Black, North Barrington, IL (US); Andy Leopold, North Barrington, IL (US); Jahi Gist, North Barrington, IL (US)

(21) Appl. No.: 17/465,113

(22) Filed: Sep. 2, 2021

Related U.S. Application Data
(63) Continuation-in-part of application No. 16/978,280, filed on Sep. 4, 2020.
(60) Provisional application No. 63/073,666, filed on Sep. 2, 2020.
(60) Provisional application No. 62/638,600, filed on Mar. 5, 2018.

(51) Int. Cl. A61B 17/12

(52) U.S. Cl. CPC A61B 17/12099; A61B 17/12136; A61B 2017/3486; A61B 2017/1205; A61B 2017/12004

(57) ABSTRACT

Devices and methods are provided for trans-esophageal aortic flow control. The device comprises a controller, an esophageal tube extending from the controller, an anchor device at a distal end of the esophageal tube and configured to anchor the distal end of the device inside a patient's stomach, and an actuator positioned proximally to the anchoring device by a sufficient distance so that the actuator will be proximal to the intersection of the patient's esophagus with their diaphragm when the anchoring device is positioned inside of the patient's stomach. In this position, the anchoring device is aligned with the location at which the patient's esophagus and aorta cross that is above (or proximal to) the intersection with the patient's diaphragm, with the patient's aorta then positioned between the spine and the esophagus. Thus, when the actuator is engaged, a compressive force is applied by the actuator against the interior of the patient's esophagus and, in turn, upon their underlying aorta so as to significantly occlude blood flow through their aorta and reduce the risk of lethal hemorrhaging from an abdominal wound.

### CROSS-REFERENCE TO RELATED APPLICATIONS

This application claims the benefit of U.S. Provisional Application No. 63/073,666 filed Sep. 2, 2020. This application is also a continuation-in-part of U.S. patent application Ser. No. 16/978,280, which application is a national stage entry of international PCT Application No. PCT/US2019/020693, which application claims the benefit of U.S. Provisional Application No. 62/638,600. Each of the foregoing applications is incorporated herein by reference in their entireties.

### FIELD OF THE INVENTION

The present invention relates generally to methods and apparatus for the treatment of hemorrhaging, and more particularly to methods and apparatus for minimally-invasive control of aortic blood pressure to mitigate hemorrhaging, and particularly non-compressible abdominal hemorrhaging.

### BACKGROUND

Hemorrhage is a leading cause of death and severe morbidity in the United States and throughout the world. The most common cause of such mortality is trauma. In fact, non-compressible abdominal wound hemorrhage is one of the leading causes of preventable death in both civilian and military trauma patients. In trauma injuries, most early deaths are caused by hemorrhage, and according to studies occur at a median of 2.6 hours after admission. Additionally, hemorrhage is responsible for 40% of civilian trauma-related deaths, and for more than 90% of military deaths that result from otherwise potentially survivable injuries. According to some professionals, about 67.3% of deaths on the battlefield are the result of hemorrhage from a wound to the truncal area. Although there are many devices developed that stop hemorrhage, many of them are not sufficient to stop internal bleeding in certain areas, such as the abdomen.

While direct pressure and tourniquets to manage bleeding from extremity injuries has significantly improved survival, internal hemorrhage within the chest, abdomen and pelvis is not easily accessible and often will continue to bleed. Uncontrolled bleeding in the torso is referred to as non-compressible torso hemorrhage (NCTH), is not amenable to control via direct pressure, and frequently leads to hemorrhagic shock and death. There are limited clinical options to treat NCTH, with emergent surgical intervention being the best option. Studies of U.S. casualties during the wars in Iraq and Afghanistan and of civilian trauma patients confirmed that hemorrhage remained the leading cause of preventable death. Recent studies estimated that 50% of early trauma deaths were due to NCTH. In military settings nearly 90% of potentially preventable pre-hospital battlefield deaths were due to hemorrhage, while nearly 70% of those preventable deaths were caused by exsanguination from truncal injuries. A study on tourniquet use in combat injuries reported 90% survival when the hemorrhage was controlled prior to the onset of shock vs 0% when an appropriate tourniquet was never applied. Multiple studies on civilian trauma have also shown the high risk of early mortality from severe hemorrhage and the critical need for early bleeding control to prevent shock and reduce the risk of death. One study conducted showed that 31% of patients suffering from NCTH and hemorrhagic shock died within 2 hours after emergency department arrival, while an additional 12% died within the first 24 hours and 11% of such hemorrhagic shock patients died after 24 hours. Among those surviving, 39% developed infection and 24% developed organ failure. In civilian trauma earlier hemorrhage control was also associated with improved survival including a 6-fold decrease in mortality with appropriate tourniquet utilization. The critical finding is that early hemorrhage control reduces blood loss and saves lives. Unfortunately, a tourniquet cannot be applied to effectively control NCTH. The ability to control such inaccessible internal bleeding would, however, provide critical time needed to get a patient to an operating room for a life-saving surgical procedure and is an unmet clinical need.

There are a number of preexisting devices that attempt to tackle this issue but fall short of fulfilling the desired outcome. Many such devices are largely theoretical, such as the chemical expanding foam RESQFOAM (available from Arsenal Medical), which describes a chemical compound that is inserted into the wound site itself and then expands to take up the entire abdominal cavity, thus putting pressure on the damaged tissue. However, the inserted foam is not biodegradable and must be completely surgically removed prior to the surgeon sewing up the wound. This process can easily result in complications and, thus, should be avoided.

Still other devices, such as the Abdominal Aortic and Junctional Tourniquet (AAJT), are only capable of preventing blood loss in juncture and not in abdominal wounds. An AAJT places pressure around the wounded area using a large belt-like device that is fastened. While this device has been implemented to a limited extent, the AAJT has only seen real success in stopping junctural hemorrhages and not abdominal hemorrhages. Therefore, it does not do an adequate job at stopping abdominal hemorrhaging. Thus, a device and method are still required to be effective in this area and to be deployed in emergency medicine.

The most successful and prevalent device on the market currently is the REBOA catheter that is capable of consistently preventing blood loss, which essentially comprises a small gastric balloon attached to a guide wire that is inserted into the femoral artery in the thigh and then snaked up to the descending aorta where the balloon is then inflated. This process decreases the flow rate to the abdomen and thus prevents bleeding. However, because of the invasive nature of the device and its insertion into the body, the procedure can only be implemented by a surgeon in a sterile operating room, and requires time that trauma patients often do not have.

As indicated by the foregoing prior efforts, unlike wounds to the extremities, normal methods of treatment to stop bleeding such as simple compression or tourniquets are simply ineffective in abdominal wounds. These wounds often involve internal bleeding and organ damage, such that applying pressure does not reach the internal wound. Therefore, there remains a need for improved methods and devices capable of decreasing the number of preventable deaths from abdominal hemorrhage, and more particularly that are minimally invasive, that are capable of preventing flow rather than pressure the wound directly, and that may readily be used and inserted into a patient by emergency services personnel in the field.

### SUMMARY OF THE INVENTION

Disclosed herein are relatively non-invasive methods and apparatus that, with respect to certain features of an embodiment of the invention, may resolve at least some of the foregoing problems. The methods and apparatus according to certain aspects of an embodiment are configured to be easily inserted into a patient's esophagus in order to apply posterior pressure to the patient's aorta. The applied pressure from the device results in the impingement or occlusion of the aorta, such that blood flow is significantly reduced if not eliminated in the lower portion of the body, including the abdomen. This allows medical professionals to extend the life of a patient while the wound is repaired. The device and its method of use are sufficiently simple so as to not require that it be administered by a surgeon, and thus can be used by many health professionals.

In certain configurations, methods and devices as disclosed herein are minimally invasive, are configured to prevent flow rather than pressure the wound directly, and are capable of insertion by emergency services in the field.

A device configured in accordance with certain aspects of an embodiment can be used by a wider range of medical personnel than previously known abdominal hemorrhage control devices due to its ease of use and non-invasiveness. This allows for using the device in locations other than operating rooms. There are many patients that could benefit from a device configured in accordance with such aspects of the invention, such as soldiers in the battlefield or patients admitted to hospitals due to injuries related to gunshots or stabbing.

A device according to certain aspects of an embodiment includes an esophageal tube and an actuator. In certain configurations, at least a portion of the actuator may be situated in a sleeve. In certain configurations, the device may include an anchor-like component, such as at least one balloon (e.g., a gastric balloon) to secure placement of the actuator and/or esophageal tube within the patient.

In accordance with certain aspects of an embodiment, the device may use magnets as the actuator to apply a force inside the body. In Magnets in Medicine, the author reviews how magnets have been widely used in medicine, and are safe to use as long as the proper precautions are taken. Before using medical devices with magnets, a medical professional should clear the area of metals that may interact with the magnetic field, and consult the patient about any devices, such as pacemakers, that may have an interaction. Magnets provide a non-contact force that can be used internally in difficult to reach locations, such as the aorta. The force of a magnet decreases with distance away from the magnet, such that the ideal specifications of the magnet are important to consider for each medical application.

In accordance with further aspects of an embodiment, a trans-esophageal aortic flow control device and method may be provided offering a portable assembly that offers a low risk safety profile with high efficacy and life-saving capabilities compared to typical devices. The device may be used by nurses, medics, and field personnel at the site of injury in pre-hospital or pre-operative settings. The device may enable many additional personnel to rapidly intervene, start resuscitation and control catastrophic bleeding earlier in forward field positions and in hospitals prior to surgical hemostasis. Such a lightweight and portable therapeutic device for early intervention by an increased number of providers to control NCTH can support those in austere environments, such as the warfighter on or near the battlefield, to reduce the amount of preventable death from hemorrhage.

In certain configurations, the device comprises a controller, an esophageal tube extending from the controller, an anchor device at a distal end of the esophageal tube and configured to anchor the distal end of the device inside a patient's stomach, and an actuator positioned proximally to the anchoring device by a sufficient distance so that the actuator will be proximal to the intersection of the patient's esophagus with their diaphragm when the anchoring device is positioned inside of the patient's stomach. In this position, the anchoring device is aligned with the location at which the patient's esophagus and aorta cross that is above (and proximal to) the intersection with the patient's diaphragm, with the patient's aorta then positioned between the spine and the esophagus. Thus, when the actuator is engaged, a compressive force is applied by the actuator against the interior of the patient's esophagus and, in turn, upon their underlying aorta so as to significantly occlude blood flow through their aorta and reduce the risk of lethal hemorrhaging from an abdominal wound.

In accordance with still further aspects of an embodiment, a device for trans-esophageal aortic flow control is disclosed, comprising: an esophageal tube having a distal end and a proximal end; an anchoring device adjacent the distal end of the esophageal tube and configured to secure placement of the distal end of the esophageal tube in a patient's stomach; and an actuator configured to apply a compressive force posteriorly in the patient's esophagus in a direction of the patient's aorta at a location in the patient's aorta that is proximal to the patient's diaphragm to at least partially occlude the patient's aorta at that location.

In accordance with still further aspects of an embodiment, a device for trans-esophageal aortic flow control is provided, comprising: an esophageal tube having a distal end and a proximal end; an anchoring device adjacent the distal end of the esophageal tube and configured to secure placement of the distal end of the esophageal tube in a patient's stomach; and an actuator configured to apply a compressive force posteriorly in the patient's esophagus in a direction of the patient's aorta, wherein the actuator is positioned on the esophageal tube proximally to the anchoring device by a sufficient distance to cause the actuator to be aligned with a portion of the patient's esophagus that is distal to an intersection of the patient's esophagus and the patient's diaphragm when the anchoring device is positioned inside of the patient's stomach.

In accordance with still yet further aspects of an embodiment of the invention, a method for trans-esophageal aortic flow control is provided, comprising: providing a trans-esophageal aortic flow control device comprising an esophageal tube having a distal end and a proximal end, an anchoring device adjacent the distal end of the esophageal tube and configured to secure placement of the distal end of the esophageal tube in a patient's stomach, and an actuator configured to apply a compressive force posteriorly in the patient's esophagus in a direction of the patient's aorta at a location in the patient's aorta that is proximal to the patient's diaphragm to at least partially occlude the patient's aorta at that location; inflating the anchoring device inside of the patient's stomach; and extending the actuator from the esophageal tube to contact the interior of the patient's esophagus so as to compress the patient's aorta at a location that is distal to the patient's diaphragm.

Still other aspects, features and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized. The present invention is illustrated by way of example, and not by way of limitation, in the figures of the accompanying drawings, in which like reference numerals refer to similar elements, and in which:

FIG. 1 is an anatomical drawing indicating the typical position of a human esophagus, aorta, and spine.

FIG. 2 is a drawing of a gastric balloon that may be used to ensure proper placement of a device as described herein and provide lateral stability.

FIG. 3 is a schematic view of a device for occluding a patient's aorta in accordance with certain aspects of an embodiment of the invention.

FIG. 4 shows exemplary dimensions of the device for occluding a patient's aorta of FIG. 3.

FIG. 5 is a schematic view showing a method for inserting and locating the device of occluding a patient's aorta of FIG. 3 inside of the patient's esophagus.

FIG. 6 is a schematic view of a trans-esophageal aortic flow control device for occluding a patient's aorta in accordance with certain aspects of an embodiment of the invention.

FIG. 7 is a side perspective view of a distal end of the trans-esophageal aortic flow control device of FIG. 6 according to further aspects of an embodiment of the invention.

FIG. 8 is a side perspective view of a distal end of the trans-esophageal aortic flow control device of FIG. 6 according to still further aspects of an embodiment of the invention.

FIG. 9 is a cross-sectional view of the trans-esophageal aortic flow control device of FIG. 6.

FIG. 10 is a side partial sectional view of the trans-esophageal aortic flow control device of FIG. 6.

FIG. 11 is a side perspective view of a section of the trans-esophageal aortic flow control device of FIG. 6 according to still further aspects of an embodiment of the invention and including a deployed covering positioned over a compression balloon.

FIG. 12 is a side perspective view of the section of the trans-esophageal aortic flow control device of FIG. 11 in which the covering is in a collapsed position.

FIGS. 13(a), 13(b), and 13(c) are schematic views of a wire fin being deployed from the trans-esophageal aortic flow control device of FIG. 6 in accordance with further aspects of the invention.

FIG. 14 is a side schematic view of wire fins being deployed from the trans-esophageal aortic flow control device of FIG. 6 in accordance with further aspects of the invention.

FIG. 15 is a perspective view of an internal shaft of an esophageal tube for use in the device of FIG. 6 and in accordance with still further aspects of the invention.

FIG. 16 is a cross-sectional view of the internal shaft of FIG. 15.

FIGS. 17(a) and 17(b) are top and bottom perspective views, respectively, of a bending base element for use in the internal shaft of FIG. 15.

FIGS. 18(a) and 18(b) are top and bottom perspective views, respectively, of a transition element for use in the internal shaft of FIG. 15.

FIGS. 19(a) and 19(b) are top and bottom perspective views, respectively, of a curved element for use in the internal shaft of FIG. 15.

FIGS. 20(a) and 20(b) are top and bottom perspective views, respectively, of a rigid shaft element for use in the internal shaft of FIG. 15.

### DETAILED DESCRIPTION OF THE PREFERRED EMBODIMENTS

The following detailed description is provided to gain a comprehensive understanding of the methods, apparatuses and/or systems described herein. Various changes, modifications, and equivalents of the systems, apparatuses and/or methods described herein will suggest themselves to those of ordinary skill in the art.

Descriptions of well-known functions and structures are omitted to enhance clarity and conciseness. The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. Furthermore, the use of the terms a, an, etc. does not denote a limitation of quantity, but rather denotes the presence of at least one of the referenced items.

The use of the terms “first”, “second”, and the like does not imply any particular order, but they are included to identify individual elements. Moreover, the use of the terms first, second, etc. does not denote any order of importance, but rather the terms first, second, etc. are used to distinguish one element from another. It will be further understood that the terms “comprises” and/or “comprising”, or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Although some features may be described with respect to individual exemplary embodiments, aspects need not be limited thereto such that features from one or more exemplary embodiments may be combinable with other features from one or more exemplary embodiments.

Provided herein are methods and devices that are configured to provide a short-term solution to major hemorrhagic bleeding to prevent extreme blood loss. For example, methods and devices in accordance with certain aspects of an embodiment can be used prior to admission to an emergency facility, while the patient is in the field, and prior to entering an operating room. Thus, the devices and methods disclosed herein are configured to:
Reduce the aortic blood flow rate by up to approximately 90% through applying radial pressure to the aorta to substantially occlude the aorta. This will prevent blood from getting to the wound and, therefore, stop the hemorrhage.
Impinge and/or occlude the aorta by inserting the device into the esophagus to compress the aorta from the patient's esophagus.

The device according to certain aspects of an embodiment includes an esophageal tube and an actuator. At least a portion of the actuator may be positioned within a sleeve. Further, the device may include an anchor, such as at least one balloon (e.g., a gastric balloon) configured to secure placement of the actuator and/or esophageal tube within the patient.

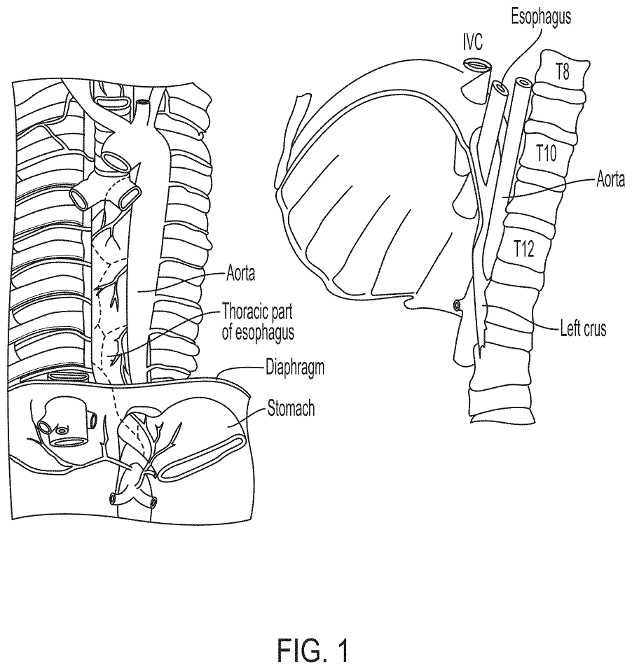

Considering the anatomy of the site of interest, and as shown in FIG. 1 (reproduced from The McGraw-Hill Companies, Inc., copyright 2006), the esophagus and the aorta cross above the intersection with the diaphragm. At this site, the aorta is “sandwiched” between the spine and the esophagus. Thus, a device configured as described herein can be inserted into the esophagus through the mouth to this location, and used to apply posterior pressure against the aorta and toward the patient's spine, which pressure will impinge upon and/or occlude the aorta.

The pressure applied to the aorta can be directed towards the posterior side of the body, instead of applying pressure in all directions, to advantageously apply the force on the aorta itself and limit unnecessary stretching of the esophagus. Total aortic occlusion is common practice in many medical procedures that involves clamping the aorta. Clamping the aorta to occlude the aorta may require an external pressure of at least 10 times the internal pressure of the aorta. For example, if an internal aortic pressure is 80 mmHg, an external pressure of 800 mmHg would need to be applied. The required force for this pressure is estimated to be about 15 lbs. However, applying pressure slightly greater than 15 lbs. would not be expected to cause any problems. The device according to certain aspects of an embodiment is preferably less than 4.5 cm in diameter so that it may be easily inserted through the mouth. This diameter is estimated based on other devices that can be inserted through the patient's mouth, however, other diameters that fit into a patient's mouth are feasible.

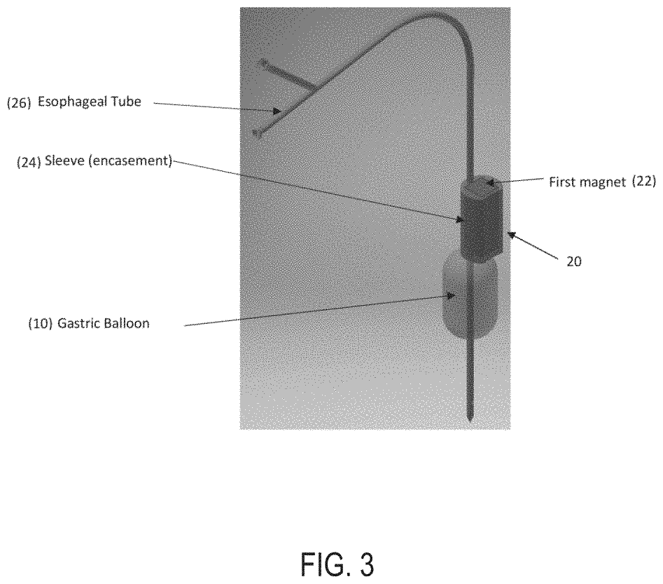

As discussed in detail below, a device according to certain aspects of an embodiment includes at least one actuator to apply a force onto a patient's aorta. The actuator is configured to control the direction of the force that is applied to the patient's esophagus, and in turn their aorta. With reference to FIGS. 3-5, the actuator may in one embodiment include one or more magnets. For example, a first magnet may be a small internal magnet and a second magnet may be an external electromagnet. The actuators, such as the magnets, can be positioned to direct forces in a desired direction and with a desired intensity or amplitude (i.e., to control the force). As discussed in further detail below, the actuator may also comprise other mechanisms that apply an occluding force on the aorta, including pneumatic (e.g., symmetric or asymmetric balloons) or hydraulic forces, and mechanical mechanisms (e.g., caused by a pulley or lever arm, a scissor-like mechanism, rigid or semi-rigid catheter-like mechanisms, stent-like mechanisms, and the like), and combinations of the foregoing. The magnitude of force can be controlled to further ensure efficiency of the device. There should generally be enough pressure to occlude the aorta, but the pressure should generally be controlled so that it does not damage internal structures such as the aorta, esophagus, and the spine.

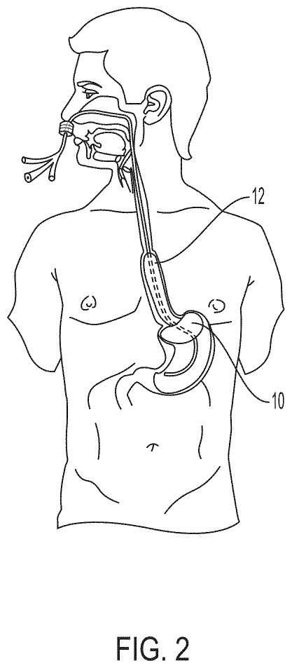

One embodiment of the device is configured to be more easily inserted and placed at the site of interest than typical devices. For example, and with reference to FIG. 2, a device and method can be used similar to that of the Sengstaken-Blakemore tube. One such embodiment may include a gastric balloon 10 that expands in the stomach to ensure the first balloon 12, or other portions of the device, are in the proper place and not in the stomach. Thus, a device in accordance with certain aspects of an embodiment may include a secondary (stomach or gastric) balloon 10 to ensure that the device is in the desired location and has not gone too far down the patient's esophagus and into the patient's stomach. A device in accordance with certain aspects of an embodiment may also include an esophageal tube for gastric content aspiration to remove the gastric contents from the patient's stomach to reduce the likelihood that the patient will vomit during use of the device.

A device formed in accordance with certain aspects of an embodiment is generally formed of simple materials. As shown in FIG. 3, the device 20 may, in accordance with certain aspects of an embodiment, include two magnets as described above, for example, a first magnet 22 that is positioned in the device (e.g., positioned in the esophageal tube (e.g., from MedEx Supply)) that is relatively smaller and/or weaker than a second magnet. Exemplary magnets may be readily commercially obtained, by way of non-limiting example, from K&J Magnetics, Inc. A device according to certain features of an embodiment may further include a sleeve 24 that can be manufactured according to typical methods, such as by additive manufacturing using CAD drawing designs. The sleeve is configured to be semi-rigid or flexible, such that it is formed of many typical materials, such as Ninjaflex material.

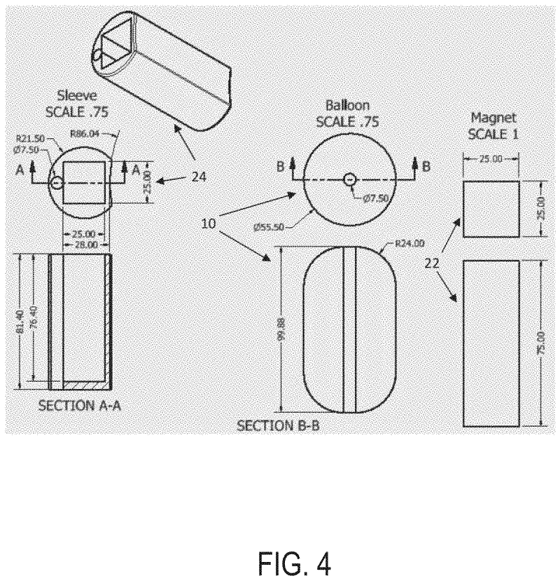

The device according to certain aspects of an embodiment can be assembled by placing the magnet in the sleeve and attaching the sleeve to an esophageal tube 26. In some embodiments, the sleeve 24 can be modified to secure the first magnet 22. Thus, one embodiment of the device includes the first (internal) magnet 22, the sleeve 24, the esophageal tube 26, the second (external) magnet (not shown), and other assembly tools (e.g., sandpaper, scissors, and fasteners or adhesive such as glue). In FIG. 3, an assembled device 20 is shown including an esophageal tube 26, a magnet 22 positioned within an encasement 24, and a gastric balloon 10, and FIG. 4 provides exemplary dimensions for such device.

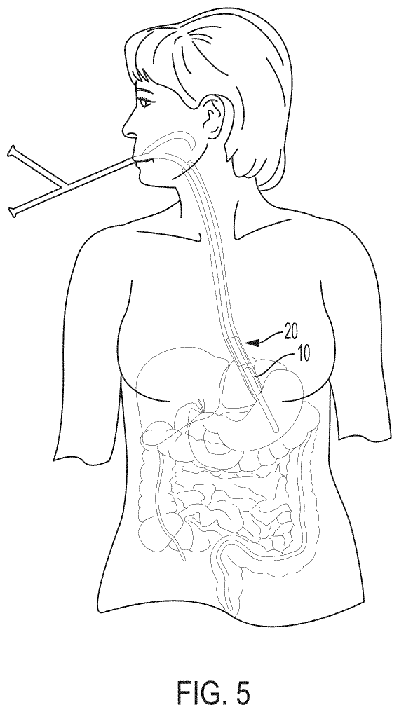

Testing of a device configured as above can include preliminary testing on an artificial model of the human aorta and esophagus. The artificial model can include a hard plastic spine, flexible plastic aorta, and flexible plastic esophagus. The artificial aorta can be filled with a fluid to mimic the pressure in the aorta. The device can be placed into the artificial esophagus, and the magnets positioned to test the ability of the magnets to occlude the aorta through the esophagus (i.e., induce an occluding force on the aorta by positioning the first and second magnet). FIG. 5 illustrates one embodiment of a method for inserting and locating the device in a patient.

Additional testing may include animal testing and/or human (e.g., cadaver) testing. For example, the testing results can be used for submission to regulatory organizations (e.g., FDA) for approval. One embodiment of the device is a Class 3, life-sustaining device. Thus, devices configured in accordance with aspects of the invention may require premarket approval before clinical tests can begin and possibly require an Investigational Device Exemption to allow testing of a high risk device. Further, devices configured in accordance with aspects of the invention may be tested in pigs. For example, sections of pig esophagus and aorta may be used to test the device on the relevant tissues, as discussed above. As discussed above, the esophageal tube can be purchased from typical medical device suppliers. In one embodiment of the device, at least one of the first or second magnets is an electromagnet. In another embodiment, the first or second magnet is a large (e.g., 4 in.×4 in.×½ in.) N52 magnet (e.g., as the second or external magnet). In one embodiment, the first (internal) magnet can be a smaller (3 in.×1 in.×1 in.) N52 magnet. In some embodiments, the magnets are encased in plastic to improve the safety of the device.

In such configurations, the use of an electromagnet to control the applied force may also allow for a tunable force for each patient that can be modified for the patient's size and blood pressure.

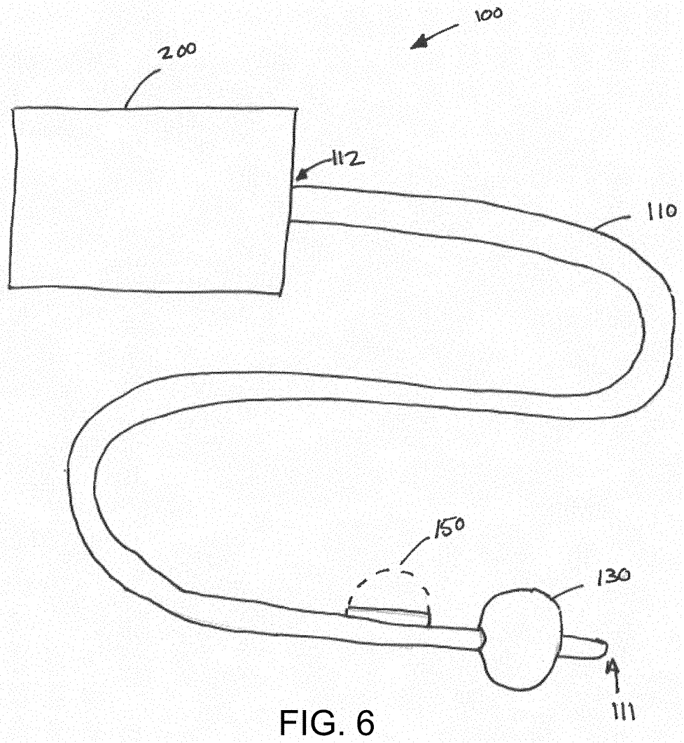

Next, and in accordance with certain features of a particularly preferred embodiment of the invention and with reference to FIGS. 6-14, a trans-esophageal aortic flow control device 100 may include an esophageal tube 110, an anchoring device 130, and an actuator 150, wherein the device 100 is configured to at least partially occlude a patient's aorta. Actuator 150 is configured to apply force posteriorly in the esophagus in a direction of the patient's aorta, with a force sufficient to at least partially occlude the patient's aorta. The esophageal tube 110 has a distal end (shown generally at 111) and a proximal end (shown generally at 112). Proximal end 112 of esophageal tube 110 is joined to (and optionally detachable from) a controller 200, which in certain configurations may comprise a hand-held controller. As discussed in greater detail below, controller 200 may provide actuators and/or connectors (all of standard configuration known to and/or readily configurable by those of ordinary skill in the art) enabling the flow of inflating gases or fluids to anchoring device 130 and/or actuator 150, suction to esophageal tube 110, and transmission of mechanical control signals (i.e., movement of mechanical members to modify and control movement, configuration, and deployment of actuator 150 and/or esophageal tube 110).

With regard to an aspect of the invention, anchoring device 130 is positioned near the distal end 111 of esophageal tube 110, and actuator 150 is positioned proximal to anchoring device 130. Anchoring device 130 may comprise a balloon, such as a gastric balloon that may be formed by way of non-limiting example of silicone, that secures the placement of the distal end 111 of esophageal tube 110 inside of the patient's stomach with anchoring device 130 inside of the stomach adjacent the gastro-esophageal junction. This will ensure that, when inflated, anchoring device 130 will not retract into the patient's esophagus from their stomach when the device is in use. Confirmation of proper placement of anchoring device 130 may be obtained through auscultation over the stomach of air injected through a dedicated air channel extending through esophageal tube 110 to distal end 111.

With respect to a particular aspect of the invention, actuator 150 is positioned proximally to anchoring device 130 by a sufficient distance so that the actuator 150 will be proximal to the intersection of the patient's esophagus with their diaphragm when the anchoring device 130 is positioned inside of the patient's stomach as detailed above. In this position, the anchoring device 150 is optimally positioned at a location at which the esophagus and the aorta cross that is above and proximal to the intersection with the patient's diaphragm, with the patient's aorta then positioned between the spine and the esophagus. Of course, those skilled in the art will readily recognize that anatomies will differ from patient to patient based at least on their size, such that a trans-esophageal aortic flow control device 100 configured in accordance with aspects of the invention may be provided in differing sizes with differing specific dimensions provided for standard internal physiology of patients of differing sizes and/or ages. Thus, the particular distance between anchoring device 130 and actuator 150 may be selected to provide such positioning with respect to the patient's aorta and diaphragm based on that standard physiology for a particular patient's size group or age group.

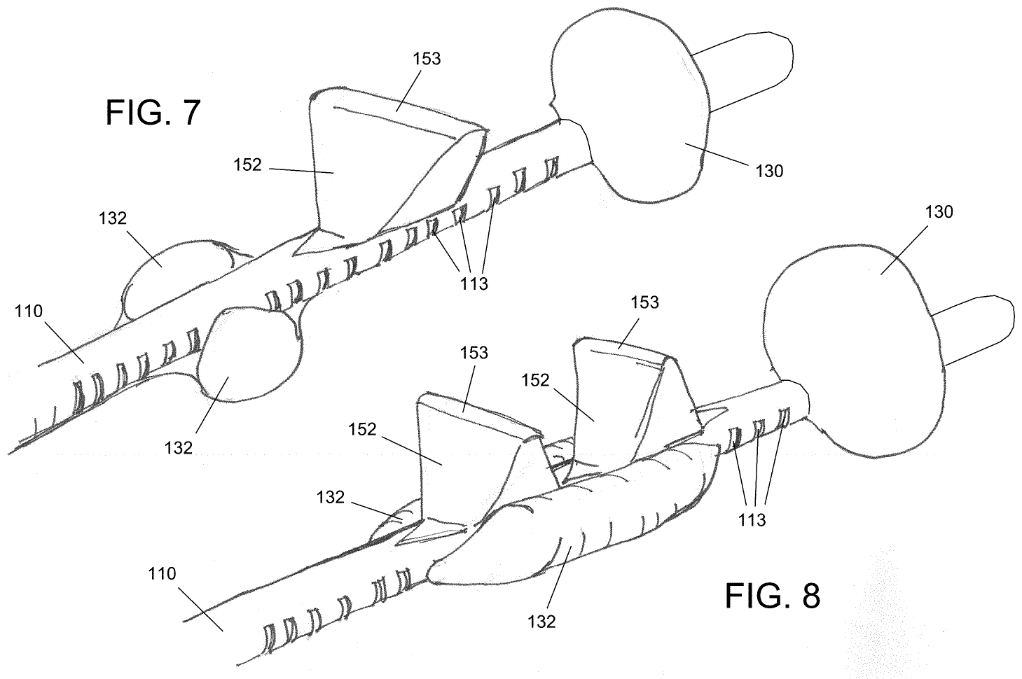

In addition to anchoring device 130 near distal end 111 of esophageal tube 110, additional proximal anchoring devices (discussed in greater detail below and shown in FIGS. 7 and 8) that are deployable and retractable from esophageal tube from a position proximal to anchoring device 130 may be provided to laterally stabilize the trans-esophageal aortic flow control device 100 in the patient's esophagus and/or cover a portion of the aortic diameter. Thus, device 100 may be configured to expand laterally to cover the entire aortic diameter to increase proximal aortic control compared to typical devices. For example, such proximal anchoring devices may be inflated using a fluid to contact the sidewalls of the patient's esophagus to laterally stabilize device 100 inside of the patient's esophagus by reducing rotation and/or translation of the device within the esophagus and with respect to the patient's aorta. In further exemplary configurations, such additional anchoring devices may comprise mechanical assemblies (e.g., extendable portions pushed by rods, wires, screws, cams, or pivots, or similarly configured mechanical operators or drivers) configured to extend contact with the sidewalls of the patient's esophagus, as further detailed below.

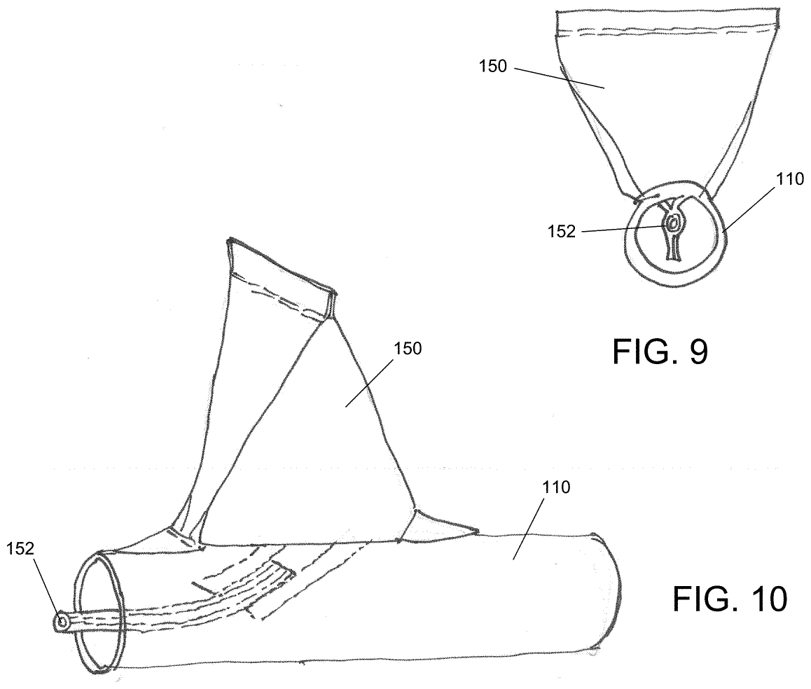

Next and with particular reference to FIGS. 7 and 8, actuator 150 may take the form of one or more compression balloons 152 that may be inflated to expand from the outer wall of esophageal tube 110 in the direction of the patient's aorta. As shown in the cross-sectional view of FIG. 9 and the side partial sectional view of FIG. 10, a conduit configured to carry inflation gas or fluid from controller 200 to compression balloon 152 may provide such inflation medium into balloon 152 when trans-esophageal aortic flow control device 100 is in position with anchoring device/gastric balloon 130 inflated inside of the patient's stomach. Such inflation medium causes compression balloon 152 to extend from the outer wall of esophageal tube 110 to push against the patient's esophagus and, in turn, compress the patient's aorta. When not inflated, compression balloon 152 is configured to sit in a collapsed position against the outside of esophageal tube 110. In certain configurations and as shown in FIG. 8, a plurality of such compression balloons 152 may be provided to expand the region of compression that is applied to the patient's aorta. Further, a top edge 153 of each compression balloon may form a thin, optionally rounded edge that is significantly more narrow than the base of each such balloon 152, and that forms a narrow line that is generally transverse to the longitudinal axis of esophageal tube 110. Such configuration of balloons 152 apply compressive force to the patient's esophagus, and thus to their aorta, along a concentrated straight line extending transverse to the direction of blood flow in the patient's aorta, thus further assisting in occlusion of the patient's aorta. Optionally, one or more additional compression balloons (not shown) may be provided between adjacent compression balloons 153 that may provide a longitudinal compression surface between the two straight line compression balloons, thus further enhancing the occlusion of the patient's aorta.

With continued reference to FIGS. 7 and 8 and as mentioned briefly above, in addition to compression balloons 153, proximal anchoring balloons 132 may also be provided which may be inflatable using an inflation fluid conduit similar in configuration to conduit 152 and supplied from controller 200. Proximal anchoring balloons 132 may be inflated after device 100 has been positioned in the patient's esophagus at the intended position with gastric anchor balloon 130 inside of the patient's stomach as described above. When inflated, such proximal anchoring balloons 132 may serve to laterally stabilize the trans-esophageal aortic flow control device 100 in the patient's esophagus and/or cover a portion of the aortic diameter. As shown in FIG. 7, such proximal anchoring balloons 132 may be positioned proximal to compression balloons 152, or as shown in FIG. 8, such proximal anchoring balloons 132 may be positioned alongside compression balloons 153. In each case, proximal anchoring balloons 132 are positioned on esophageal tube 110 so as to, when inflated, apply a force against the patient's esophagus in a direction that is different from the direction of force application from compression balloons 152, and more preferably are orthogonal to the direction of application of the compression force from compression balloons 152.

As shown in FIGS. 7 and 8, esophageal tube 110 may include perforations or ports 113 configured to allow fluid flow between the interior of tube 110 and the exterior of tube 110. Thus, when suction is applied at proximal end 112 of esophageal tube 110, any fluids inside of the patient's stomach and/or esophagus may be evacuated through esophageal tube 110.

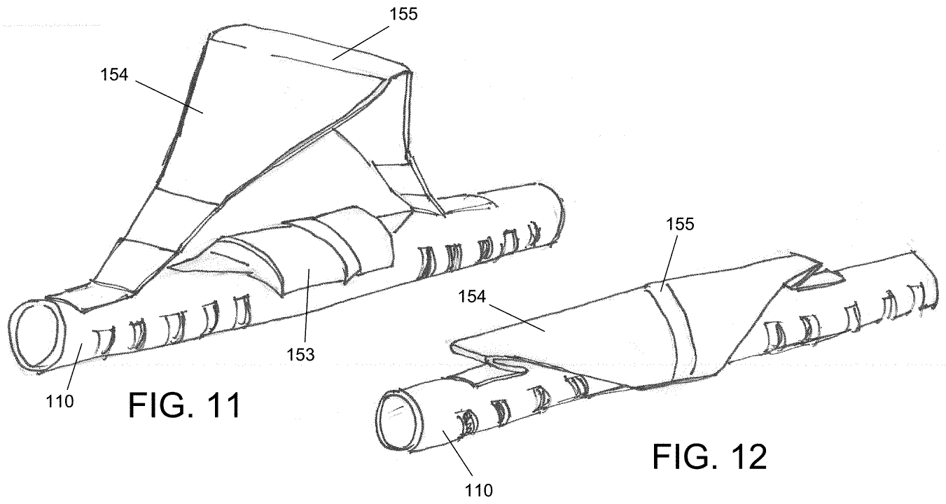

Optionally in certain configurations, a compression balloon cover 154 may be provided over a compression balloon 153 as shown in FIGS. 11 and 12, with compression balloon cover 154 configured with a narrow, top edge 155. Compression balloon cover 154 may be formed with creases causing it to take the shape shown in FIG. 11 when fully extended (upon inflation of compression balloon 153), thus forming narrow top edge 155 extending transverse to the longitudinal axis of esophageal tube 110 and, thus, transverse to the direction of blood flow through the patient's aorta. When compression balloon 153 is uninflated, compression balloon cover 154 takes the form shown in FIG. 12 in which it is collapsed against uninflated compression balloon 153 and the exterior of esophageal tube 110.

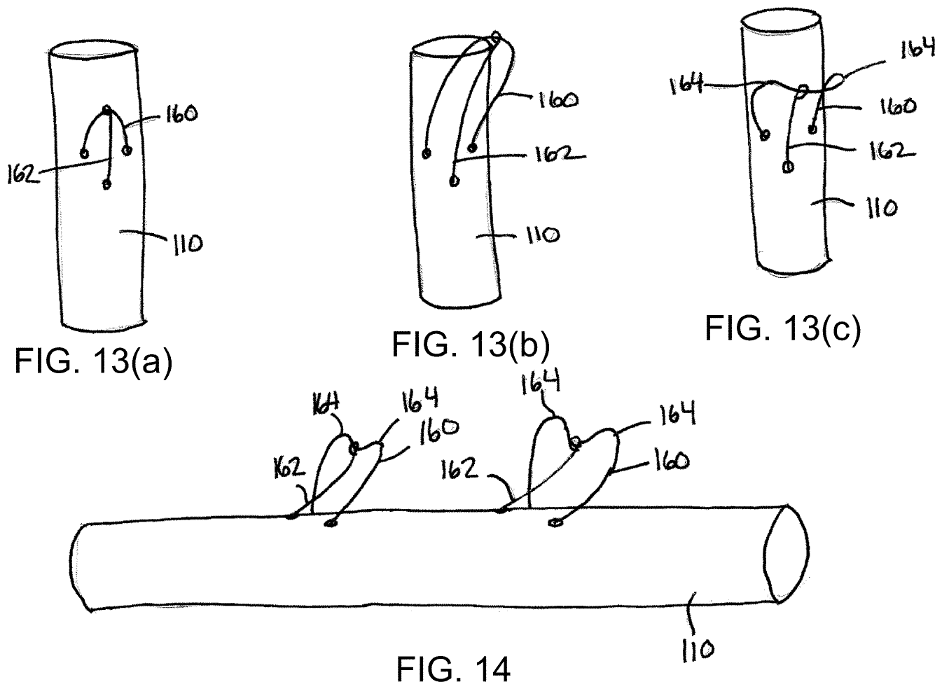

In certain configurations, actuator 150 may further comprise wire fins 160, which in a particularly preferred configuration may be comprised of Nitinol wires that deploy to their intended fin shape when fully deployed from esophageal tube 110. As shown in FIGS. 13(a)-13(c), a stiff actuation wire 162 may be attached at its distal end to a wire fin 160, and at its opposite proximal end to controller 200. When actuation wire 162 is pushed from actuator 200 towards distal end 111 of esophageal tube 110, the attached wire fin 160 likewise extends outward from esophageal tube 110. FIG. 13(a) shows a wire fin 160 in a retracted position, while FIG. 13(b) shows wire fin 160 in a partially deployed position as it extends outward from esophageal tube 110. As wire fin 160 reaches its fully deployed position shown in FIG. 13(c), the top, outer portion of wire fin 160 assumes its memory shape to form outwardly extending wings 164 on opposite, lateral sides of each wire fin 160. As shown in FIG. 13(c) and the side view of device 100 of FIG. 14 with fully deployed wire fins 160, the extended wings 164 of each wire fin 160 may likewise compress the patient's esophagus against their aorta again along a line of force application that is transverse to the longitudinal axis of esophageal tube 110, with the extended wings 164 pushing laterally outwardly to expand the region of the patient's esophagus that engages their aorta. While FIG. 14 shows two such wire fins being deployed from esophageal tube 110, those skilled in the art will recognize that any number of wire fins 160 configured as discussed herein may likewise be provided.

In certain configurations, actuator 150 may comprise in combination wire fins 160 and one or more compression balloons 153 as detailed above, thus forming a dual pneumatic and mechanical mechanism to provide the required directional esophageal compression over the length and width of the underlying aorta. In certain configurations of such dual actuator configurations, wire fins 160 may be positioned outside of compression balloons 153, such as on opposing longitudinal sides of each compression balloon 153, or alternatively one or more wire fins 160 may be positioned inside of a compression balloon 153, all without departing from the spirit and scope of the invention. By way of non-limiting example, a thin, inflatable polyurethane balloon may enclose one or more such wire fins 160. Such a balloon may likewise be formed of higher durometer polyurethanes, silicone, and Pebax. In other configurations, fins 160 may be positioned outside and at opposite ends of balloon 153. The particular dimensions of the balloon and the deployable fins are preferably selected to maximize the diameter and length of aortic compression while minimizing the space that is required within the shaft 170 of esophageal tube 150 to accommodate the deployable fins 160.

An internal wire mechanism extends through esophageal tube 110 and is configured for stiffening, steering, and stabilizing trans-esophageal aortic flow control device 100 once in the intended position with actuator 150 located to compress the patient's aorta. Such internal wire mechanism provides improved steerability so that device 100 can more efficiently be moved into position to occlude the patient's aorta. As further detailed below, the rigidity of an internal shaft 170 of esophageal tube 110 may be controlled using such wire mechanism to allow greater flexibility for navigating the patient's oropharynx and esophagus, while also providing sufficient rigidity to enable compression of the patient's aorta during the deployment of the actuator 150. In an exemplary prototype configuration, the elements of the shaft 170 of esophageal tube 110 were created using Stratasys Vero White 3D printing material with an internal wire that could be actuated to provide stiffening as detailed below. Those skilled in the art will readily recognize that the elements of shaft 170 may likewise be formed through a dedicated design molding process using specific materials that balance their properties with the foregoing requirements for both stiffness and flexibility. In certain preferred configurations, such materials may comprise (by way of non-limiting example) cross-linked polyethylene (PEX), simple polyethylene, and Nylon.

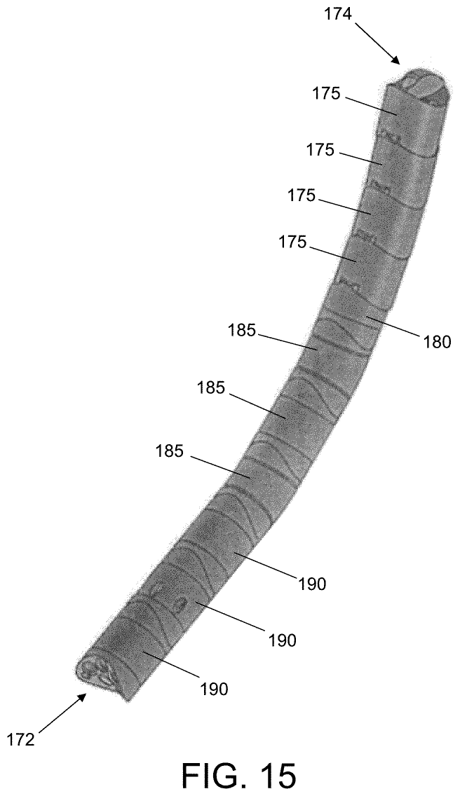

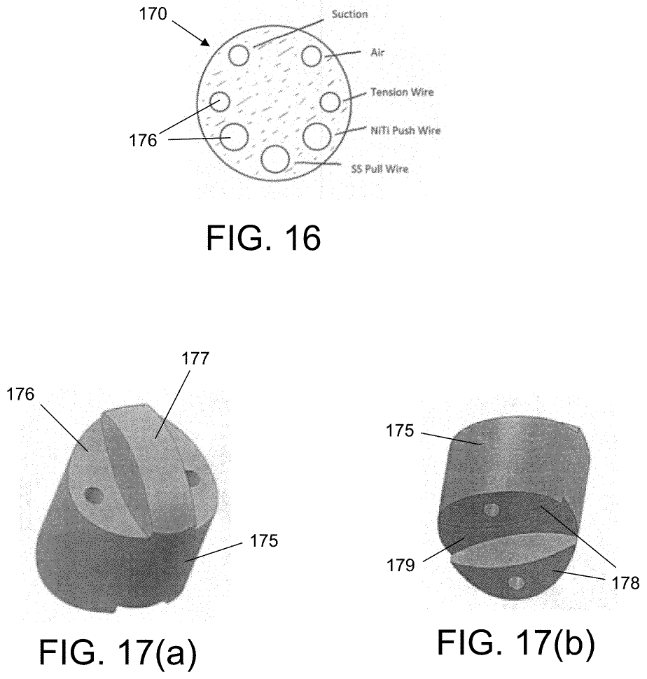

FIG. 15 shows a manipulable, variable flexibility and steerable shaft 170 in accordance with further aspects of the invention, which may form the primary structure of esophageal tube 110. Shaft 170 (which may optionally be enclosed within a sheath) has a distal end 172 closest to actuator 150 and anchor device 130, and a proximal end 174 that may be joined to controller 200. Shaft 170 is preferably formed by a series of segments that may be at least partially articulated with respect to one another so as to aid in steering device 100 to its intended location within the patient's esophagus. Further and as shown in the cross-sectional view of shaft 170 of FIG. 16, shaft 170 may include a plurality of channels 176 extending through the length of shaft 170 (and thus through each articulating segment of shaft 170) to provide for each of suction through esophageal tube 110, delivery of air to anchor device 130 and actuator 150, tension wires for varying the stiffness of shaft 170, and control wires 162 for deployable fins 160. Tensioning wires (not shown) may extend from controller 200 through shaft 170 and may be retracted or tightened by controller 200 to pull them taught, in turn pulling distal segments of shaft 170 in the direction of controller 200 and causing the assembly to stiffen. When tension is released from such wires, shaft 170 may sit in a more flexible configuration with its articulating segments in turn allowing shaft 170 to conform to the patient's internal physiology as shaft 170 is moved into position inside of their esophagus.

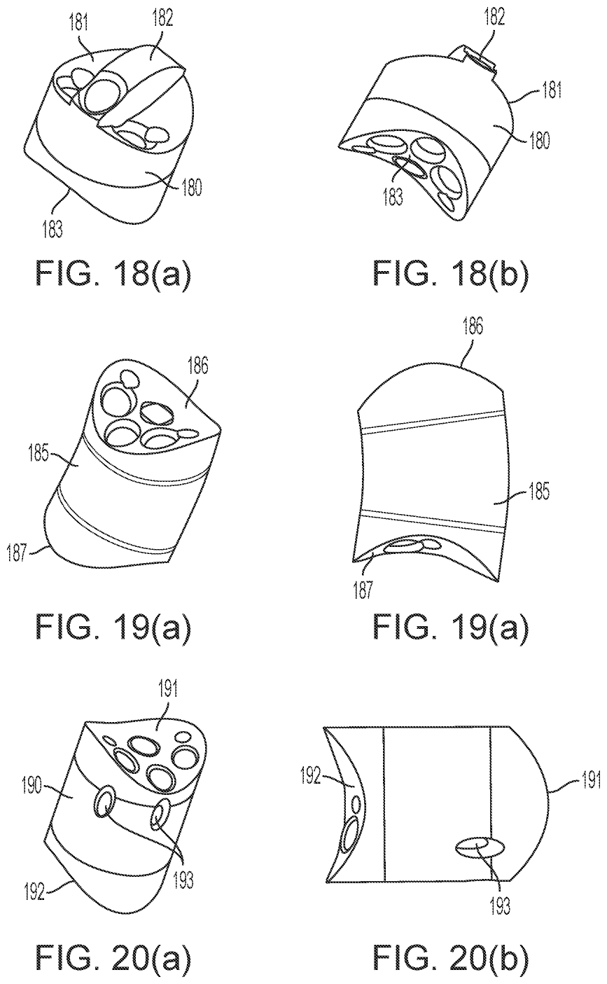

With continuing reference to FIG. 15, shaft 170 may, at its proximal portion starting at proximal end 174, form a bendable portion of shaft 170 that may curve when tension is released from the tensioning wires extending through shaft 170. Such bendable portion of shaft 170 may be formed by a series of bending base elements 175 as shown in FIGS. 17(a) and 17(b), each of which has a top concave face 176 and a curved joint wall 177, and a bottom convex face 178 having a curved notch 179. Each top concave face 176 is formed complementary to bottom convex face 178, and each curved joint wall 177 is formed complementary to curved notch 179. With this configuration, adjacent bending base elements 175 may pivot with respect to one another to enable bendable portion of shaft 170 to bend during placement within the patient's esophagus. Distal to the bending portion is a transition element 180, as shown in FIGS. 18(a) and 18(b). Transition element 180 has an upper concave face 181 and curved joint wall 182 for pivotally mating with the adjacent bending base element 175, and has a lower concave face 183 for mating with a complementary face of curved elements 185, as shown in FIGS. 19(a) and 19(b), each having complementary and mating top faces 186 and bottom faces 187 enabling them to be joined together in a fixed orientation with respect to one another providing a centrally, fixedly curved portion of shaft 170. Next, the distal most covered element 185 abuts a rigid shaft portion of shaft 170 comprising rigid shaft elements 190 as shown in FIGS. 20(a) and 20(b), each having complimentary and mating top faces 191 and bottom faces 192 enabling them to be joined together in a fixed orientation with respect to one another providing a distal, fixed straight portion of shaft 170. One or more of rigid shaft elements 190 preferably include openings 193 for extension of actuation wires 162, wire fins 160, and any other portions of actuator 150 as may be desirable in a particular implementation.

In cadaveric testing, a trans-esophageal aortic flow control device 100 configured in accordance with the foregoing disclosure was inserted through the esophagus and into the stomach with placement confirmed by auscultation of air insufflation through a dedicated gastric port in esophageal tube 110. Anchoring device 130 in the form of a distal gastric balloon was inflated, and the device 100 secured at the gastro-esophageal junction. The compression mechanism of actuator 150 was deployed and found capable of achieving near complete trans-esophageal aortic occlusion at the diaphragmatic hiatus and partial occlusion of the more proximal thoracic aorta.

As will be clear to those of ordinary skill in the art from the foregoing disclosure, abdominal hemorrhage control presents a major unmet clinical need. By controlling the aortic flow in the descending part of the aorta proximal to the patient's diaphragm, devices and methods configured in accordance with aspects of the invention will substantially prevent blood flow to the lower chest and abdomen. This will significantly reduce blood loss and extend the life of the patient long enough to allow for a surgeon to access and repair the wound area. Devices and methods configured in accordance with aspects of the invention are less invasive and easier to implement for aortic occlusion than typical methods, such as REBOA, and thus offer significant improvement over previously known devices and methods.

Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. Thus, it should be understood, therefore, that the invention may be practiced otherwise than as specifically set forth herein.

What is claimed is:

1. A device for trans-esophageal aortic flow control, comprising:
an esophageal tube having a distal end and a proximal end;
an anchoring device adjacent the distal end of the esophageal tube and configured to secure placement of the distal end of the esophageal tube in a patient's stomach; and
an actuator configured to apply a compressive force posteriorly in the patient's esophagus in a direction of the patient's aorta at a location in the patient's aorta that is proximal to the patient's diaphragm to at least partially occlude the patient's aorta at said location.

2. The device of claim 1, wherein said actuator is positioned on said esophageal tube proximally to the anchoring device by a sufficient distance to cause the actuator to be aligned with a portion of the patient's esophagus that is distal to an intersection of the patient's esophagus and the patient's diaphragm when the anchoring device is positioned inside of the patient's stomach.

3. The device of claim 1, said actuator further comprising a compression balloon extensible from an exterior of said esophageal tube.

4. The device of claim 3, said compression balloon having a body that narrows from the esophageal tube to a top edge of the compression balloon.

5. The device of claim 4, wherein the top edge of the compression balloon extends transverse to a longitudinal axis of the esophageal tube.

6. The device of claim 4, further comprising a plurality of compression balloons, and a longitudinal compression balloon positioned between at least two of said compression balloons.

7. The device of claim 3, further comprising a cover extending over and extendable upon inflation of said compression balloon, wherein said cover defines a top edge extending transverse to a longitudinal axis of the esophageal tube.

8. The device of claim 1, said actuator further comprising a wire fin extensible from the esophageal tube.

9. The device of claim 8, said wire fin further comprising outwardly extending wings defining a compression edge extending transverse to a longitudinal axis of the esophageal tube.

10. The device of claim 1, said anchoring device further comprising a gastric balloon.

11. The device of claim 10, further comprising a plurality of proximal anchoring devices positioned adjacent or proximal to the actuator and configured to laterally stabilize the esophageal tube in the patient's esophagus.

12. The device of claim 11, wherein said proximal anchoring devices further comprise balloons extensible in a direction that is orthogonal to a direction of force application from the compression balloon.

13. The device of claim 1, said esophageal tube further comprising an internal shaft comprised of a plurality of articulable segments.

14. The device of claim 13, wherein said articulable segments are configured to engage one another so as to allow at least portions of the internal shaft to bend.

15. The device of claim 14, said device further comprising internal tension wires attached to segments of said internal shaft and configured to change a stiffness of said internal shaft in response to tensioning said internal wires.

16. A device for trans-esophageal aortic flow control, comprising:
an esophageal tube having a distal end and a proximal end;
an anchoring device adjacent the distal end of the esophageal tube and configured to secure placement of the distal end of the esophageal tube in a patient's stomach; and
an actuator configured to apply a compressive force posteriorly in the patient's esophagus in a direction of the patient's aorta, wherein said actuator is positioned on said esophageal tube proximally to the anchoring device by a sufficient distance to cause the actuator to be aligned with a portion of the patient's esophagus that is distal to an intersection of the patient's esophagus and the patient's diaphragm when the anchoring device is positioned inside of the patient's stomach.

17. A method for trans-esophageal aortic flow control, comprising:
providing a trans-esophageal aortic flow control device comprising an esophageal tube having a distal end and a proximal end, an anchoring device adjacent the distal end of the esophageal tube and configured to secure placement of the distal end of the esophageal tube in a patient's stomach, and an actuator configured to apply a compressive force posteriorly in the patient's esophagus in a direction of the patient's aorta at a location in the patient's aorta that is proximal to the patient's diaphragm to at least partially occlude the patient's aorta at said location;
inflating the anchoring device inside of the patient's stomach; and
extending the actuator from the esophageal tube to contact the interior of the patient's esophagus so as to compress the patient's aorta at a location that is distal to the patient's diaphragm.

18. The method of claim 17, wherein said actuator further comprises a compression balloon extensible from an exterior of said esophageal tube.

19. The method of claim 17, wherein said actuator further comprises a wire fin extensible from the esophageal tube, said wire fin having outward extending wings defining a compression edge extending transverse to a longitudinal axis of the esophageal tube.

20. The method of claim 17, wherein said trans-esophageal aortic flow control device further comprises a plurality of proximal anchoring devices positioned adjacent or proximal to the actuator and configured to laterally stabilize the esophageal tube in the patient's esophagus.
